(19) United States
(12) Patent Application Publication
(10) Pub. No.: US 2022/0061715 A1
(43) Pub. Date: Mar. 3, 2022

(54) Stacked Oximeter and Operation Method

(71) Applicant: Senbiosys, Neuchâtel (CH)

(72) Inventors: Assim Boukhayma, Neuchâtel (CH); Antonino Caizzone, Milvignes (CH)

(21) Appl. No.: 17/411,168

(22) Filed: Aug. 25, 2021

Related U.S. Application Data
(60) Provisional application No. 63/070,436, filed on Aug. 26, 2020.

(51) Int. Cl. A61B 5/1455; H01L 25/04

(52) U.S. Cl. CPC A61B 5/14552; A61B 2562/0238; H01L 25/043

(57) ABSTRACT

A stacked photoplethysmography (PPG) sensor for oximetry is capable of sensing simultaneously, with optimal area and quantum efficiency, PPG signals using a plurality of emission wavelengths without the need for time division multiplexing.

### RELATED APPLICATIONS

This application claims the benefit under 35 U.S.C. § 119(e) of U.S. Provisional Application No. 63/070,436, filed on Aug. 26, 2020, which is incorporated herein by reference in its entirety.

### BACKGROUND OF THE INVENTION

Nowadays, wearable devices, such as fitness trackers or smartwatches, with optical heart rate sensors, are becoming common.

The technology behind these sensors is called photoplethysmography (PPG), which is an optical measurement technique used to detect blood volume changes in living tissues. A PPG sensor requires few optoelectronics components, such as a light source, e.g. light-emitting-diode (LED) to illuminate the living tissue, a photodetector (PD) to track any light intensity variation due to the blood volume change through the cardiac cycle and an analog front-end (AFE) for signal conditioning and processing. Today, the importance of PPG for medical monitoring is proven by the number of primary vital signs directly or indirectly recordable out of it.

The PPG signal is obtained by shining light from the LED at a given wavelength, in the visible or near-infrared range, into a human tissue, e.g. finger, wrist, forehead, ear lobes. The PPG sensor or photodetector detects the light transmitted through (transmissive PPG) or reflected (reflective PPG) from the tissue and transforms it into a photogenerated current. The detected signal, i.e. PPG signal, has two different components: a large DC (quasi-static) component corresponding to the light diffusion through tissues and non-pulsatile blood layers, and a small AC (pulsatile) part due to the diffusion through the arterial blood. The AC component is only a very small fraction (typically 0.2% to 2%) of the DC one, meaning the AC component is 500 to 50 times smaller than the DC component. This mostly depends on the body location and the LED wavelength and weakly on the skin tone. Such small AC/DC ratio is often called perfusion-index (PI).

The hemoglobin plays a key role in transporting the oxygen via the red blood cells. Specifically, one hemoglobin molecule can carry up to four oxygen molecules and, in this case, it is usually named as oxygenated hemoglobin (HbO2). The HbO2 features different light properties with respect to the de-oxygenated hemoglobin (Hb), as shown in FIG. 1. This is the mechanism exploited by a pulse oximeter to provide the oxygen saturation, also named SpO2.

Oximetry can be performed according to a number of approaches. In one case, a plurality of photonic sensors is used with optical filters and LEDs. In another case, a single wide band photonic sensor is used with a plurality of time division multiplexed LEDs.

Commercially available pulse oximeters usually embed red and near-infrared (NIR) light sources, working in time-division-multiplexing (TDM). Specifically, the pulse oximeter works out the SpO2 by comparing how much red light and NIR light is absorbed by the blood. Depending on the amounts of HbO2 and Hb present, the ratio, i.e. RoR, of the amount of red light absorbed compared to the amount of infrared light absorbed changes. Using this ratio, the pulse oximeter can then work out the SpO2, via a calibration curve:

SpO2%=k₁+k₂·RoR,

where k1 and k2 are the calibration constants. Practically, the SpO2 reports the percentage of the oxygenated hemoglobin, e.g. HbO2, with respect to the whole hemoglobin family (Hb+HbO2):

$$SpO 2 \% = 100 \cdot \frac{HbO 2}{H b + H b O 2} ,$$

The larger the SpO2 is, the more oxygenated the blood is.

The recent works, see C. Lochner, Y. Khan et A. Pierre, All-organic optoelectronic sensor for pulse, Nature Communications, vol. 5, p. 5745, 2014 and A. Caizzone, A. Boukhayma et C. Enz, A 2.6 uW Monolithic CMOS Photoplethysmographic (PPG) Sensor Operating with 2 uW LED Power for Continuous Health Monitoring, IEEE Transactions on Biomedical Circuits and Systems, 2019, have presented pulse oximeters embedding visible light LEDs only, i.e. green and red. Indeed, by looking at FIG. 1, it is clear that the difference in the extinction coefficients between Hb and HbO2 at green (˜550 nanometers (nm)) is comparable to the one at NIR(˜825 nm). In other words, it is possible to define a value RoR which determines how much red light and green light is absorbed by the blood.

Generally, employing this visible light is justified by its shallower skin penetration, which intrinsically leads to some advantages. There are disadvantages to using the visible as outlined in M. Y, M. Sekine et T. Tamura, The advantages of wearable green reflected photoplethysmography, Journal of Medical Systems, vol. 35, n %15, pp. 829-834, 2011 and W. Cui, L. E. Ostrander et B. Y. Lee, In vivo reflectance of blood and tissue as a function of light wavelength, IEEE Transactions on Biomedical Engineering, vol. 37, n %16, pp. 632-639, 1990. Indeed, the green light is the wavelength which, at a given power budget, maximizes the PI of the PPG signal. See A. Caizzone, An ultra low-noise micropower PPG sensor, EPFL PhD Thesis, 2020. Most of the medical relevant information relies on the AC component only. This is particularly important in the smartwatch segment since the wrist comes with quite limited PI values. This is the reason why commercially available smartwatches often integrate green emitters for heart rate monitoring. In addition, thanks to its lower penetration, the green light shows a larger resilience to motion-artefacts (MA).

### SUMMARY OF THE INVENTION

On the other hand, the shallower skin penetration can suffer from poor performance at low temperatures, when it is important to shine deeper to reach thicker arteries. Better penetration is achievable by the NIR.

An oximeter combining the advantages of visible and NIR operations is key towards better and more versatile Sp⁰² monitoring.

At the same time, energy and area are key parameters for wearable PPG sensors particularly for an ear or finger-worn device. Indeed, such a device must feature extremely small form factor together with low energy consumption.

Moreover, the implementation of monolithic CMOS PPG sensors embedding the photo-sensing part as well as the processing part in a same silicon die seems to be the optimal approach for miniaturizing the PPG sensing devices. It is difficult, however, to conceive CMOS optical sensors with high performance in both visible and NIR wavelengths.

Thus, this invention relates to the ever-growing field of health monitoring and particularly oximetry. It concerns a device and operating technique often allowing the extraction of the blood oxygen saturation with optimum power consumption, minimum area and high fidelity by operating in the visible and NIR.

In general, according to one aspect, the invention features a photoplethysmography (PPG) sensor system, comprising stacked silicon optical sensor chips having different thicknesses.

In a current embodiment, the stacked silicon optical sensor chips comprise three stacked silicon optical sensor chips. Each of these optical sensor chips is often mounted on a glass substrate. Further, the top of the optical sensor chips is preferably less than 10 μm thick, the middle of the optical sensor chips is less than 100 μm thick, and the bottom of the optical sensor chips is greater than 100 μm thick.

A controller is typically provided to determine an oxygen saturation from green, red and infrared signals from the optical sensor chips.

In general, according to one aspect, the invention features a photoplethysmography (PPG) sensing method. This comprises detecting light with stacked silicon optical sensor chips having different thicknesses, resolving green, red and infrared signals from the optical sensor chips, and determining an oxygen saturation from green, red and infrared signals from the optical sensor chips.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a plot of the extinction coefficients for oxyhemoglobin (HbO2) and deoxyhemoglobin (Hb) as a function of a function of the wavelength in nanometers;

FIG. 2 is a plot of silicon light absorption depth as a function of the wavelength;

FIG. 3 is a plot of photon intensity as a function of the depth in silicon for blue (450 nm), green (550 nm) and red (650 nm);

FIG. 4 is a schematic diagram showing a stack of image sensors of different thicknesses and the example of red, green and red values derivation from a stack of three silicon-on-glass image sensors with the respective silicon layer thicknesses;

FIG. 5 is a schematic side view of a stacked sensor system according to the present invention for oximetry with its package;

FIG. 6 shows an operation method to extract a more robust and SpO2 value from a PPG signal based on the present invention.

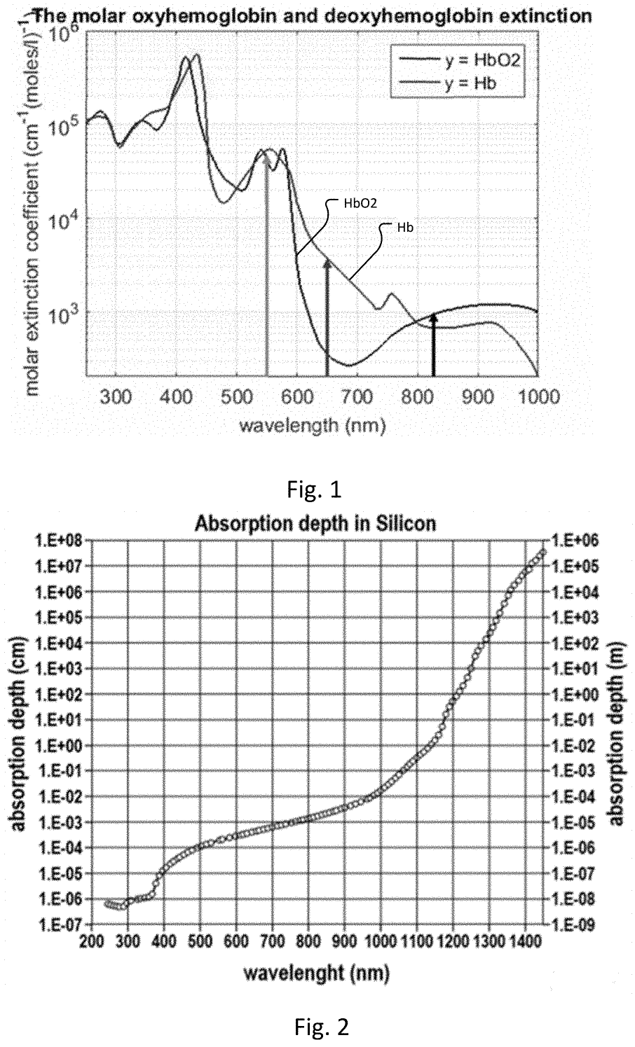

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The light absorption in silicon is subject to the Beer Lambert law. The light intensity at a depth L in the silicon corresponds to:

I(L)=I0e−●(●)L,

where 1/α(λ) is the absorption depth in silicon for a wavelength λ.

FIG. 2 shows the measured dependence of α on the wavelength. It demonstrates that the thickness of the silicon chip can be used to filter photons based on their wavelength. For instance, a 2 micrometer (μm) thick silicon is almost fully absorbing 450 nm wavelength while remaining about 60% transparent to 650 nm wavelengths.

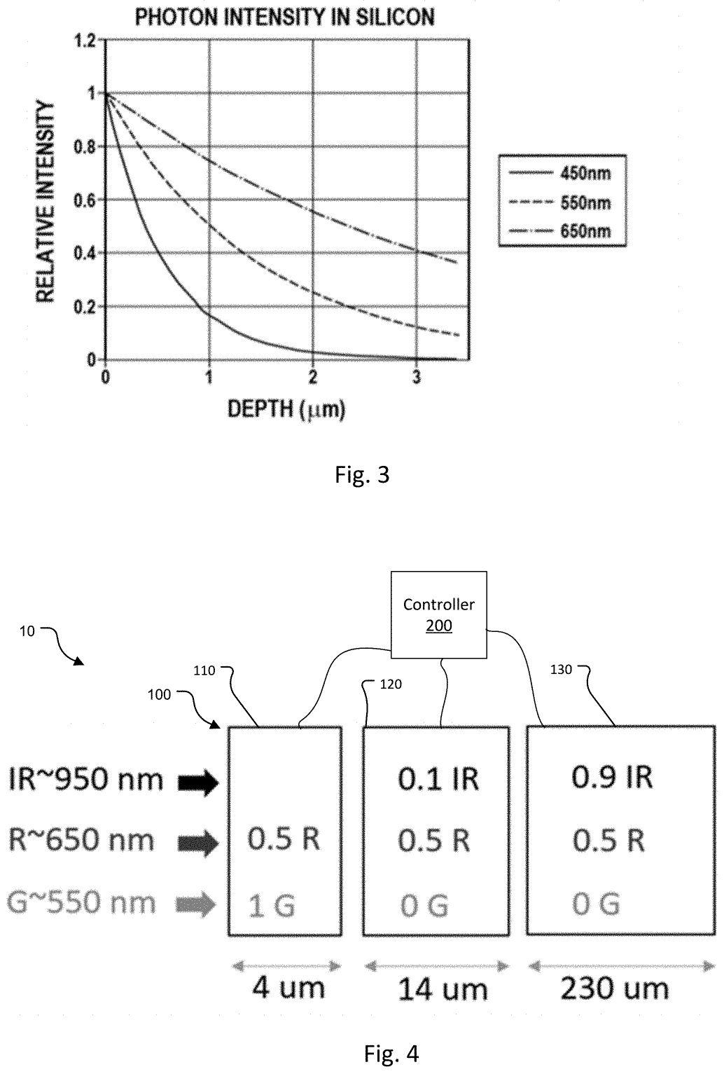

FIG. 3 is a plot of the photon intensity as a function of the depth in silicon for blue (450 nm), green (550 nm) and red (650 nm).

At the same time, the thinning process of silicon chips and particularly silicon image sensor chips have been dramatically improved during the last decade. The main motivation for chip thinning today is the use of back-side illumination as well as chip stacking.

For example, in recent generations of image sensor chips, transistors with metal layers and micro-lenses with color filters are formed on opposite sides of a back-side illuminate (BSI) chip. In this way, the quantum efficiency is significantly improved since metal layers and in-pixel transistors do not interact with the incident light but rather reflect back part of the light not absorbed by silicon to the photo diode such as a pinned photo diode (PPD).

Chip stacking has also improved. In one example, a back side illuminated CMOS Image Sensor (CIS) chip is stacked with another chip dedicated for the digital processing. The two chips' metal layers are connected with deep through-silicon vias (TSVs). In this way, the pixel analog circuitry and logic circuitry can be separated, not only in two chips, but also in two different technology nodes.

The present approach preferably involves replacing time-multiplexed LEDs or a plurality of sensors to perform oximetry by a plurality of stacked silicon sensors having each a cleverly chosen thickness so that they absorb a specific range of wavelengths. Such an implementation features the following advantages: small size, low energy consumption, and low optical loss and improved NIR performance.

Based on the light absorption properties of silicon, one can think of vertically stacking CMOS photonic sensors having each a chosen substrate thickness as the most efficient way to sense different spectral components of a wide band photonic light flux without multiplexing sensors or using color filters.

FIG. 4 shows an illustration of this principle with three optical and specifically image sensor chips 110, 120, 130 in a stacked sensor system 100 of a photoplethysmography (PPG) sensor system 10. The optical sensors 110, 120, 130 each have a different silicon layer thickness. By exploiting the dependence of the absorption depth on the wavelength, one can choose the thickness for each photonic sensor chip.

In one exemplary embodiment, the optical sensor chips are each image sensor chips that each comprise a two-dimensional array of pixels such as greater than 100 by 100 pixel array. That said, in other embodiments, the optical sensor chips include a smaller number of pixels such as single pixels or a linear array of 5 or more pixels.

In the example, most of the green spectra gets absorbed in the top image sensors 110 featuring a thickness of less than 10 μm and usually less than 6 μm or preferably about 4 μm thick. The red component is split between the top image sensors 110 and the middle image sensor 120, which middle sensor has a thickness of less than 100 μm and usually less than 30 μm or preferably about 14 μm thick. The bottom image sensor 130 with a thickness of greater than 100 μm or preferably about 230 μm thick. This bottom image sensor collects only the NIR component.

This stack of sensors 100 allows a controller 200 to perform PPG signal detection and oxygen saturation analysis. Specifically, the controller resolves and records with green, red and NIR wavelengths from the patient and uses these wavelengths to determine oxygen saturation for the patient at the same time and using a minimum area.

Fabrication Method

A preferred fabrication method of such a stacked sensor system 100 involves a wafer front-end back-end and packaging processing steps allowing stacking multiple layers of photonic sensors having each a different silicon thickness and a transparent substrate allowing the light not absorbed in one sensor to be absorbed in the next ones.

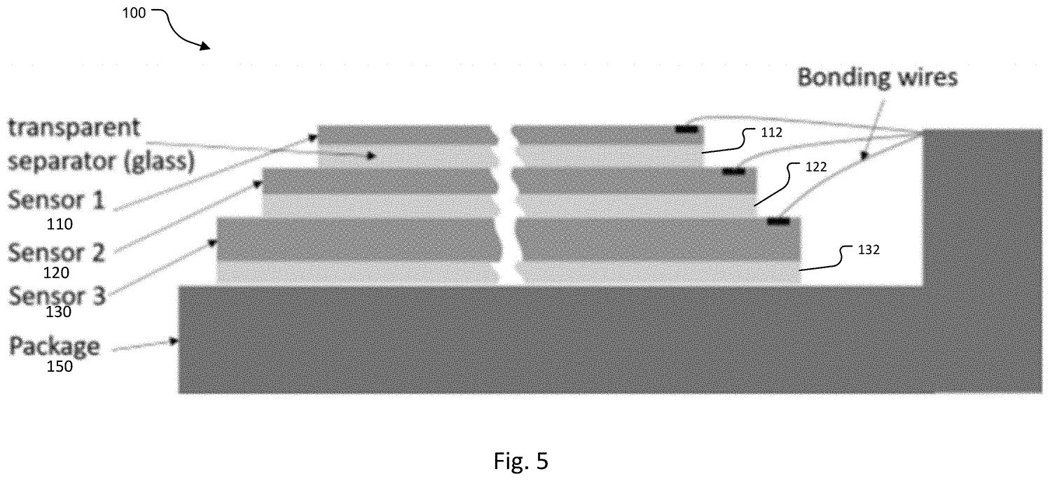

FIG. 5 shows one embodiment of the stacked sensor system 100.

The fabrication method can start from a conventional silicon wafer and a glass wafer (or a wafer made of a transparent material that can be bonded to silicon) for each of the sensors 110, 120, 130. The silicon and glass wafers are first cleaned and bonded. Anodic bonding can be used here, for instance, in a way that does not introduce any intermediate layer keeping the interface fully transparent to light. The obtained silicon to glass wafer is then thinned from the silicon side to reach the desired silicon thickness (the importance of this step comes from the fact that it is very difficult to manipulate very thin wafers, hence bonding them to glass wafers can allow achieving any silicon thickness while avoiding handling issues). The thinned silicon-on-glass wafer is then processed into photodiodes, electronic circuitry, metal layers and microlens layers in a conventional way. Multiple silicon-on-glass sensor wafers can be processed in this way with different silicon layer thickness. These wafers can then be stacked and then diced or the sensor dies can also be stacked after dicing.

FIG. 5 shows an example of stacked sensor system 100 with three stacked sensors in a package using wire bonding.

In the example, the top, thinnest CMOS sensor 110 is bonded to its glass substrate 112. This is stacked on the middle CMOS sensor 120, which has its own glass substrate 122. The glass substrate 122 of the middle CMOS sensor 120 is bonded to the top of the bottom CMOS sensor 130. The bottom sensor 130 is bonded to a package 150 by its glass substrate 132. Wire bonds can then be made from the package 150 to the respective sensors 110, 120, 130.

Operation Method

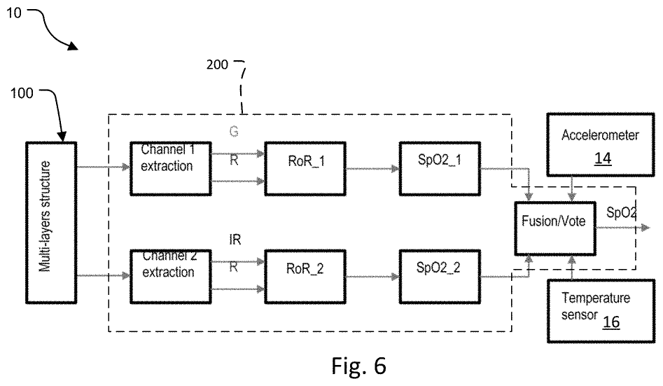

FIG. 6 shows the operation method performed by the controller 200 based on the information from the stacked sensor system 100. This allows the controller 200 to effectively combine different PPG channels towards a better SpO2 extraction. The method allows PPG signal recordings with green, red and NIR wavelengths to be performed at the same time and using a minimum area.

It has also been disclosed that, for the given thicknesses, such structure leads to most of the green spectra getting absorbed in the top sensor 110, while the red component is split between the top sensor 110 and the middle sensor 120. On the contrary, the majority of the NIR is collected by the bottom sensor 130. For this reason, and particularly for the red, it is important to recover the integrity of its incident emission.

In this regard, a first block which gets as inputs the output of each silicon layers and properly establishes, by simple mathematical subtractions or additions, the right value for each of the three emitting wavelengths. Once the correct values are established, i.e. green (G), red (R) and infrared (IR), then two separate and independent channels are processed. In channel 1, the visible components, G and R, are used to compute RoR_1, where RoR is a ratio of ratios.

As a general rule, by computing AC and DC from a PPG signal, the change in absorption of light in atrial blood is determined. This is caused by blood pumping from the heart, with no contribution from other tissue.

The ratio of the AC component to the DC component is known as the perfusion index, which is the ratio of the pulsating blood flow to the nonpulsatile static blood flow. The goal of a PPG-based heart rate or SpO2 measurement system is to increase the AC to DC signal ratio, where the perfusion index is PI=AC/DC.

The perfusion index for green and red wavelengths can be used to calculate the ratio of ratios (RoR).

Similarly, channel 2 embeds R and IR which are exploited to compute RoR_2, which yields perfusion index for infrared and red wavelengths. As a reminder, depending on the amount of HbO2 with respect to Hb, RoR changes, this happens for the two channels, independently. The two RoR values are eventually converted into the SpO2, by the means of two different calibration curves.

In addition, the photoplethysmography (PPG) sensor system 10 further includes an accelerometer 14 and a temperature sensor 16 for monitoring patient motion and the patient's skin temperature.

In normal operations, meaning under little or no motion artifacts (MA) as measured by the accelerometer 14 and room temperature as measured by the temperature sensor 16, the two channels will likely give rise to very close SpO2 values. Outside those cases, the two processing channels implemented by the controller 200 may compute different SpO2 values. This is intrinsically linked to the way the PPG signal behaves in the presence of low temperatures or large MA. See Y. Maeda, M. Sekine et T. Tamura, Relationship between measurement site and motion artifacts in wearable reflected photoplethysmography, Journal of Medical Systems, vol. 35, n %15, pp. 969-976, 2011. In this regard, it is important to combine the two channels smartly to increase the confidence level of the measurement. Specifically, the system employs both the temperature sensor 16 and the accelerometer 14. Under regular temperature and acceleration operations, the two SpO2 values are simply fused by the controller 200 and the final extracted SpO2 corresponds to the mean of each channel. On the contrary, under large MA or low temperature operations, the final extracted SpO2 corresponds to one of the two channels, according to a voting mechanism employed by the controller 200. The fusion/vote mechanism is automatically and continuously activated throughout the oximeter operations.

The industrial applications relate to wearable consumer electronic devices such as smartwatches, wrist bands, ear buds and smart rings. This is also particularly relevant under pandemic situations during which portable devices tracking respiratory systems can provide key information to the health care system.

This invention is also of direct interest to medical applications in which oximetry is largely exploited under different ways such as medical patches or medical bands to be used during clinical stays or for patient post monitoring (at home).

While this invention has been particularly shown and described with references to preferred embodiments thereof, it will be understood by those skilled in the art that various changes in form and details may be made therein without departing from the scope of the invention encompassed by the appended claims.

What is claimed is:

1. A photoplethysmography (PPG) sensor system, comprising stacked silicon optical sensors chips having different thicknesses to resolve green, red, and infrared signals.

2. The sensor system according to claim 1, wherein the stacked silicon optical sensors chips comprise three stacked silicon optical sensors chips.

3. The sensor system according to claim 1, wherein each of the optical sensor chips is mounted on a respective glass substrate.

4. The sensor system according to claim 1, wherein a top of the optical sensor chips is less than 10 μm thick.

5. The sensor system according to claim 1, wherein a middle of the optical sensor chips is less than 100 μm thick.

6. The sensor system according to claim 1, wherein a bottom of the optical sensor chips is greater than 100 μm thick.

7. The sensor system according to claim 1, wherein the optical sensor chips are image sensor chips.

8. The sensor system according to claim 1, further comprising a controller determining an oxygen saturation from green, red and infrared signals from the optical sensor chips.

9. A photoplethysmography (PPG) sensing method comprising
detecting light with stacked silicon optical sensor chips having different thicknesses to resolve green, red, and infrared signals;
resolving green, red and infrared signals from the optical sensor chips; and
determining an oxygen saturation from the green, red and infrared signals detected by the respective the optical sensor chips.

10. The method according to claim 9, wherein the silicon optical sensor chips are image sensor chips.

11. The method according to claim 9, further comprising three stacked silicon optical sensor chips.

12. The method according to claim 9, further comprising mounting each of the optical sensor chips on respective glass substrates.

13. The method according to claim 9, further comprising thinning a top optical sensor chip to less than 10 μm thick.

14. The method according to claim 9, further comprising thinning a middle optical sensor chip to less than 100 μm thick.

15. The method according to claim 9, further comprising providing a bottom optical sensor chip that is greater than 100 μm thick.
